(19) United States
(12) Patent Application Publication
(10) Pub. No.: US 2015/0127029 A1
(43) Pub. Date: May 7, 2015

(54) DEVICE AND METHOD FOR ANASTOMOSIS

(71) Applicant: The Trustees of Columbia in the City of New York, New York, NY (US)

(72) Inventor: Syed Tasnim Raza, New York, NY (US)

(21) Appl. No.: 14/536,193

(22) Filed: Nov. 7, 2014

Related U.S. Application Data
(63) Continuation-in-part of application No. 12/940,127, filed on Nov. 5, 2010.
Application No. 12/940,127 is a continuation of application No. PCT/US2013/040171, filed on May 8, 2013.
(60) Provisional application No. 61/644,263, filed on May 8, 2012.

(51) Int. Cl. A61B 17/115; A61B 17/11

(52) U.S. Cl. CPC A61B 17/1155; A61B 17/1152; A61B 17/11; A61B 2017/1107; A61B 2017/1132; A61B 2017/1157
USPC 606/153

(57) ABSTRACT

A device and method for anastomosis are disclosed which may include a stapler with a base and one or more stapling limbs. A graft having an asymmetrical end portion may be sleeved on the base. The apparatus includes a base having a length from a front end and at least one stapling limb disposed proximate the base, with each stapling limb having stapling functionality at its head. Each stapling limb end is configured for movement from an open position at a distance from the front end of the base to a closed position closer to the front end of the base. The front end of the base is configured to engage at least a portion of a graft for connection to a vessel, wherein the base and graft are insertable at least partially into a portion of the vessel with at least a portion of the graft, vessel and base overlapping each other. Control is configured to cause each stapling limb in the closed position to fire at least one staple into the overlapping portions of the vessel and graft to make a connection there between.

### RELATED APPLICATIONS

This application is a continuation of PCT/US2013/040171, filed May 8, 2013, which claims priority to U.S. Provisional Patent Application No. 61/644,263 filed May 8, 2012. This application is a continuation-in-part of co-pending, U.S. patent application Ser. No. 12/940,127, filed Nov. 5, 2010, the disclosures of each are hereby incorporated by reference in its entirety.

### BACKGROUND OF THE DISCLOSED SUBJECT MATTER

1. Field of the Disclosed Subject Matter

The disclosed subject matter relates generally to the field of surgery and particularly to surgical suturing devices and methods.

2. Description of Related Art

A heart is illustrated in FIG. 1, and is generally designated by the reference letter H. The heart H includes a right atrium RA, a left atrium LA, a right ventricle RV, a left ventricle LV, a pulmonary artery PA, and an aorta A. The aorta A includes an intimal (inner) layer or intima IL (FIG. 2) and an adventitial (outer) layer or adventitia AL.

In Type A aortic dissection, the intimal (inner) layer or intima IL is torn and defines a tear T in the ascending aorta AA or aortic arch. Blood B collects in (unnumbered) between the layers AL, IL. This is referred to as a false lumen. If the blood collection continues, the false lumen may block off origin of major arteries coming off the aorta, thus causing abrupt lack of blood flow to the involved organ, causing its death. This condition may be fatal, or cause of major morbidity, if not treated urgently.

Treatment may necessitate dissection of an aortic portion Ap of the aorta A containing the portion with intimal tear along lines of dissection LD1, LD2, and replacement of the cutout portion of the aorta by a Dacron® tube graft of appropriate size. More particularly, the dissection along lines of dissection LD1, LD2 may separate a distal end or end portion Ea of the ascending aorta AA from a proximal end or end portion Ed of the ascending aorta AA. The two ends or end portions Ea, Ed of the ascending aorta AA, after the dissected aortic portion Ap and particularly the intimal portion thereof has been excised, are still quite fragile and the two layers AL, IL are still separated. The two ends Ea, Ed may be prepared for the Dacron® tube graft by suturing the respective ends Ea, Ed and buttressing them with strips of Dacron® or felt both on the inside and the outside of the ends Ea, Ed. After both ends Ea, Ed are prepared, the Dacron® tube graft is anatamosed to restore the continuity of the aorta and establish blood flow.

The treatment described above may take a considerable length of time using prior art methods and devices. During the treatment, the patient is on cardiopulmonary bypass (heart-lung) machine. For part of the time, all circulation is stopped. On average, a patient may be on the heart-lung machine for three hours and 20 minutes, and the average time of a patient's circulation being stopped is 34-40 minutes. Subjecting the patient to such treatment for the above durations of time contributes to the increased risk of stroke, bleeding, and death. In particular, at the time of preparation of this material, the operative mortality rate is 17.84%, post-op neuro deficit is 10.3%, and reexploration for bleeding is 16.5%. Thus, there is need to reduce the length of time that it takes to carry out the treatment of replacing an excised portion of the aorta with a graft of appropriate size and shape, wherein the replacement technique and equipment itself does not subject the delicate vessel walls to tearing, as conventional suturing procedures are known to suffer from.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

Generally stated, the disclosed subject matter relates to apparatuses and methods for anastomosis. Exemplary embodiments provide a stapler that may be used for anastomosis such as between the end of a dissected aorta and the end of a Dacron® tube graft. Features and actions of the exemplary embodiments allow for generally faster anastomosis than with prior art devices and methods as explained below.

The disclosed subject matter may be embodied as an apparatus of a relatively small design advantageous for use in the limited space of the operative field. An exemplary device according to the disclosed subject matter may be a single unit including the base and stapling heads. The single unit design may reduce alignment complexity and problems compared to working with other devices where the base and stapling head may be separate units. The exemplary device requires only generally minimal dexterity for operation—an advantage especially in a confined space at least because the device may be actuated by simply turning or actuating a control knob. Moreover, certain embodiments of the disclosed subject matter can be configured such that a single actuation of the control mechanism can accomplish multiple staple firings.

Time savings are additional advantages. Use of the disclosed subject matter in exemplary devices and methods may save critical time during anastomosis procedures. One way in which time is saved is that the disclosed subject matter does not require “cuffing” of the end of the aorta for stapling to the graft.

Using exemplary embodiments, the user may avoid or at least minimize the use of sutures in anastomosis and the problems caused by sutures. In previous embodiments, sutures were often pulled “tight” to effect a seal, but such pulling could lead to tearing delicate tissues and further bleeding. In contrast, the multiple staples fired by exemplary embodiments into the materials to be connected form a strong leak-free connection thereof. The embodiments of staples into two rows according to the interleaved or staggered pattern may provide higher (stronger) bonding and higher leak resistance than connections made by prior art devices and methods.

To achieve these and other advantages and in accordance with the purpose of the disclosed subject matter, as embodied and broadly described, the disclosed subject matter includes an apparatus for anastomosis comprising a base having a length from a front end and at least one stapling limb disposed proximate the base, with each stapling limb having stapling functionality at its limb end corresponding to the front end of the base. Each stapling limb end is configured for movement from an open position at a distance from the front end of the base to a closed position closer to the front end of the base. Also included is a control capable of being selectively activated to cause movement of each stapling limb end between the open and closed positions. The front end of the base is configured to engage at least a portion of a graft for connection to a vessel, wherein the base and graft are insertable at least partially into a portion of the vessel with at least a portion of the graft, vessel and base overlapping each other. The control is configured to cause each stapling limb in the closed position to fire at least one staple into the overlapping portions of the vessel and graft to make a connection therebetween.

In some embodiments, the front end of the base is configured with an arcuate shape and the at least one stapling limb includes two stapling limbs disposed generally circumferentially about the front end of the base. Also, the control actuates a cam to cause movement between open and closed positions. In select embodiments, the front end of the base is received within a folded portion of the graft, such that the front end of the base has a layer of the graft on opposing first and second surfaces of the base. In such embodiments, the base and each stapling limb are configured to engage an asymmetrical graft.

In accordance with an aspect of the disclosed subject matter, the connection between the overlapping graft and vessel comprises a substantially leak-proof connection. In some embodiments each stapling limb includes multiple rows of staples which are fired into the overlapping layers of the vessel and graft forming two generally parallel rows, the staples of each row being staggered with respect to the staples of the other row. The staples from each row of each stapling limb can be fired substantially simultaneously.

In accordance with another aspect of the disclosed subject matter, a device for substantially leak-free fastening of at least two elements comprises a base having a fastening end which has first and second opposing surfaces. At least one fastening mechanism is positioned about the fastening end of the base and configured for movement between an open position about the fastening end and a firing position about the fastening end. The base is configured to receive a length of a first element disposed over the first and second opposing surfaces of the base with the fastening mechanism in the open position. The fastening end of the base is configured to be received in a portion of a second element to form an overlap between a portion of the first element, second element and fastening end of the base with the fastening mechanism in the open position. A control is configured to activate the at least one fastening mechanism to move from the open position into the firing position about the fastening end to make a substantially leak-free fastening between the first and second elements at the overlap. The at least one fastening mechanism is responsive to an opening activation to move away from the firing position to allow for withdrawal of the fastened first and second elements from the base. In some embodiments, the fastening end of the base is configured with an arcuate shape.

In accordance with another aspect of the disclosed subject matter, a method for anastomosis is disclosed comprising positioning a graft on a base of an arcuate stapler with the stapling heads of the stapler disposed in an open position relative to the base. The base is then inserted into a dissected end of an aorta so that a segment of the graft underlies a portion of the dissected end of the aorta to form an overlap between the graft and the aorta. The stapling heads are then moved into a firing position relative to the base within the overlap such that staples are fired in two parallel rows within the overlap to form a leak-free connection between the graft and the aorta. After stapling, the stapling heads are moved to the open position relative to the base and the overlap and the base is withdrawn from the graft, wherein the graft remains connected to the aorta in the leak-free connection at the overlap.

In some embodiments, positioning a graft on a base of an arcuate stapler includes forming a fold within the graft and inserting the base of the arcuate stapler into the fold. Additionally, the firing position of the stapling heads includes a radial distribution of the staple heads within the overlap, and firing the staples includes causing each staple to pierce one layer of the graft and the aorta. Typically, each staple at least partially closes interior to the overlap upon engaging with the base. Thereafter, the arcuate stapler is repositioned substantially adjacent to the location of a first firing location and fires additional staples within the overlap. Furthermore, in certain embodiments the graft has an asymmetrical shape.

More particularly, the stapling limbs of the exemplary embodiment may be disposed in pairs with each of the limb ends in a pair firing three staples in two rows into the overlap. A first limb end of the pair may fire a staple into a top row and two staples into a bottom row, the two staples in the bottom row being spaced apart, and the staple in the top row positioned generally parallel at least in part to the space between the two staples in the bottom row. A second limb of the pair may fire two staples into a top row and a staple into a bottom row, the two staples in the top row being spaced apart, and the staple in the bottom row positioned generally parallel at least in part to the space between the two staples in the top row.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

FIG. 1 is a perspective view of a human heart, and illustrates various portions thereof, including the aorta.

FIG. 2 is a highly enlarged perspective view of the aorta of FIG. 1 with portions broken away for clarity, and illustrates intimal and adventitial layers thereof, including an intimal tear in the intima or inner layer of the aorta.

FIG. 3 is a perspective view of an embodiment of a circular stapler in accordance with the disclosed subject matter.

FIG. 4 is a perspective view of the embodiment of FIG. 3 with the addition of a secured graft.

FIG. 5 is a perspective view of part of the embodiment of FIG. 3 as it may be in use in the operative field.

FIG. 6 is a perspective view of the embodiment of FIG. 3 as it may be in use in connecting a graft to an end of an aorta.

FIG. 7 is a perspective view of the embodiment of FIG. 3 with the limbs of the embodiment in a closed position.

FIG. 8 is a lengthwise cross section view of the embodiment of FIG. 7 taken along the line defined by A-A′ in FIG. 7.

FIG. 9 is a drawing of three exemplary limbs of an exemplary embodiment of the disclosed subject matter.

FIG. 10 is a radial cross section of the exemplary embodiment of FIG. 7.

FIG. 11A is an exemplary embodiment of an unformed staple that may be used with an exemplary embodiment of the disclosed subject matter.

FIG. 11B is an exemplary embodiment of a formed staple that may be used with an exemplary embodiment of the disclosed subject matter.

FIG. 12 is an illustration of exemplary results of use of an exemplary embodiment of the disclosed subject matter in connecting a graft with an end of an aorta.

FIG. 13 is a flow diagram illustrating an exemplary embodiment of the disclosed subject matter.

FIGS. 14A-B depict two exemplary limbs of an exemplary embodiment of the disclosed subject matter.

FIG. 15 is an illustration of a graft having a tubular end with a circular opening and an asymmetrical end with a non-cylindrical opening as it may be in use in connecting to an end of an aorta.

FIG. 16A is an axial view of an alternative exemplary embodiment of the disclosed subject matter.

FIG. 16B is a perspective view of the exemplary embodiment of FIG. 16A.

FIG. 17 is an illustration of exemplary results of use of an exemplary embodiment of the disclosed subject matter in connecting a graft with an end of an aorta.

FIG. 18 is an illustration of a cross-sectional view of an exemplary embodiment of the disclosed subject matter in connecting a graft with an end of an aorta.

FIG. 19 is an illustration of a cross-sectional view of a connection of a graft with an end of an aorta in accordance with an exemplary embodiment of the disclosed subject matter.

FIG. 20 is a perspective view of a handle for deploying and operating the exemplary embodiments of FIGS. 3 and 16B in accordance with an exemplary embodiment of the disclosed subject matter.

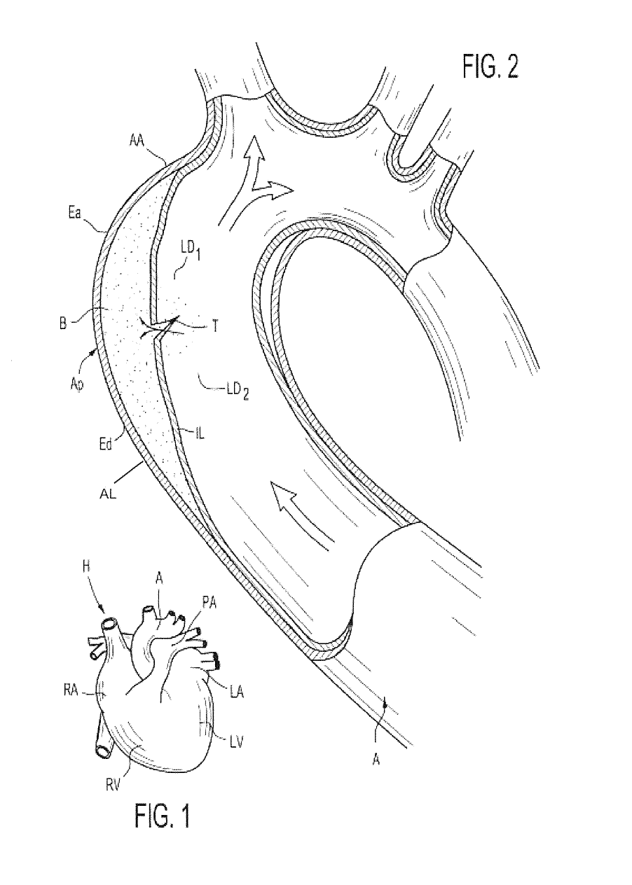

### DETAILED DESCRIPTION OF THE EXEMPLARY EMBODIMENTS

Reference will now be made in detail to the exemplary embodiments of the disclosed subject matter, examples of which are illustrated in the accompanying drawings. The method and corresponding steps of the disclosed subject matter will be described in conjunction with the detailed description of the system.

Generally stated, the disclosed subject matter relates to a device and method for making a leak-proof connection between a first element and a second element. The disclosed subject matter is described below by reference to exemplary embodiments, but the disclosed subject matter should not be limited by such embodiments or examples provided. The disclosed subject matter, however, can be embodied in many different forms and carried out in a variety of ways, and should not be construed as limited to the embodiments set forth in this description and/or the drawings. The exemplary embodiments that are described and shown herein are only some of the ways to implement the disclosed subject matter. Elements and/or actions of the disclosed subject matter may be assembled, connected, configured, and/or taken in an order different in whole or in part from the descriptions herein.

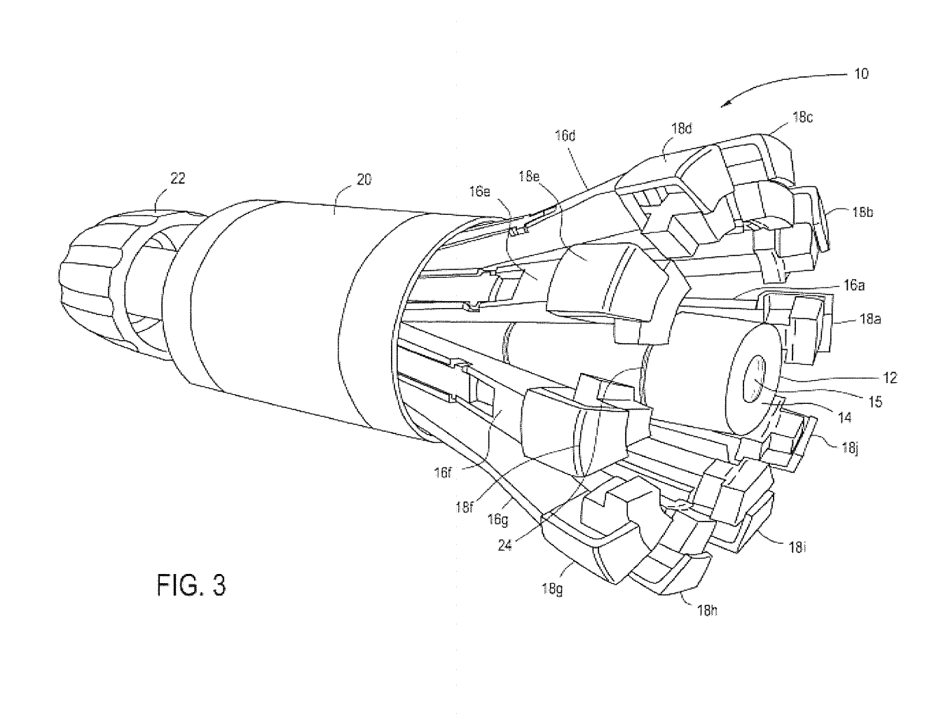

FIG. 3 illustrates an exemplary embodiment 10 of the disclosed subject matter as a device that may be used for anastomosis between the generally circular end of a dissected aorta and a tubular graft of an appropriate size. For convenience, this exemplary embodiment 10 may be referred to as a “circular aortic stapler.” An overview of the exemplary embodiment 10 is provided in the paragraph immediately below with additional details in the paragraphs that follow. The term “device” is used herein synonymously with the term “apparatus”.

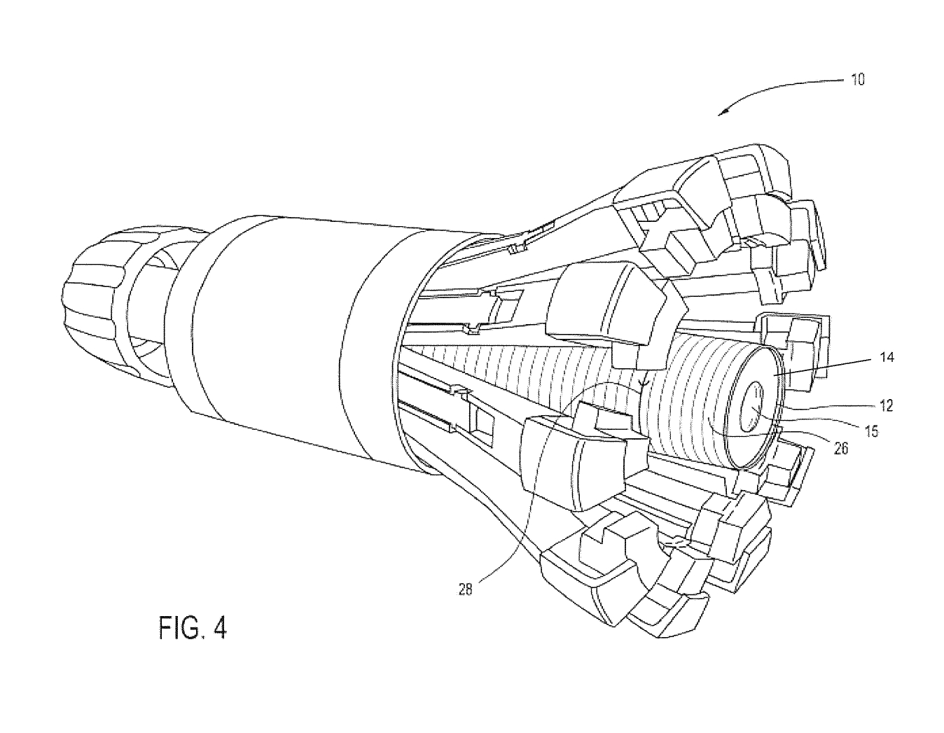

The exemplary embodiment 10 in FIG. 3 includes a central cylindrical anvil 12 with limbs 16a-16j splayed in a generally circular fashion about the front end 14 of the anvil 12 when the embodiment 10 is in the open position, such as when ready for use. (In FIG. 3, limbs 16b, 16c, 16h, 16i, and 16j are not visible.) Generally stated, to use the embodiment 10, a tubular graft 26 (not shown in FIG. 3, see FIG. 4) may be positioned over and along the cylindrical central anvil 12 of the exemplary embodiment 10. The graft 26 may be said to be “sleeved” on the anvil 12. To use the exemplary circular aortic stapler 10 in the operative field, the anvil 12 with the tubular graft 26 may be positioned inside one end of the dissected aorta so the end of the aorta lies between the splayed limbs 16a-16j of the circular aortic stapler 10 and the anvil 12 with tubular graft 26. When the exemplary embodiment is activated, the limbs 16a-16j move from their respective open positions to closer positions relative to each other and to and about the front end 14 of the anvil 12. The embodiment 10 may be further activated to cause the limbs 16a-16j to staple the end of the aorta to the tubular graft 26 at the overlap between the two on the anvil 12. The stapling effects a substantially leak-free connection between the end of the dissected aorta and the tubular graft 26. To remove the exemplary circular aortic stapler 10, its limbs 16a-16j may be caused to open and the anvil 12 may be slid from the connected aorta and graft without disturbing the leak-free connection therebetween.

More particularly described, the exemplary embodiment 10 shown in FIG. 3 includes an anvil 12 at its center. The anvil 12 may also be referred to as a base (as will be described in further detail below), or a graft support means.

In this example 10, the anvil 12 is generally cylindrical having a length from a front end 14 to a back end (not shown in FIG. 3). Also in this example, the anvil 12 includes a hollow core 15 (to at least partially accept a control screw (not shown in FIG. 3)). The anvil 12 may be made of stainless steel (and/or other appropriate material). The size of the anvil may vary depending on the size of the tubular graft to be used with the anvil. The size of the tubular graft, in turn, may vary depending on the size of the end of the dissected aorta with which the tubular graft is to be connected. Thus, the size of the anvil may vary depending on the size of the end of the dissected aorta to which the tubular graft is to be connected in a leak-proof manner.

In the exemplary embodiment 10, limbs 16a-16j are disposed about the length of the anvil 12. A limb also may be referred to as an arm, a splay arm, a fastening mechanism (or part of a fastening mechanism), or a leak proof connection means (or part of a leak proof connection means).

In this example, the limbs 16a-16j are disposed generally circumferentially about and along at least some part of the length of the anvil 12. The limb disposition also is generally symmetrical about the anvil 12 in the embodiment 10. Each limb 16a-16j may be made of stainless steel (and/or any other appropriate material). Generally, the size and length of each limb is approximately the same in this embodiment, and such features may depend on the size of the anvil 12. The exemplary embodiment 10 includes ten (10) limbs 16a-16j, but the number of limbs may vary depending upon the embodiment of the disclosed subject matter.

Each limb 16a-16j in this embodiment 10 includes a stapling head 18a-18j at a limb end corresponding to the front end 14 of the anvil 12. A stapling head may be referred to as a stapling arm or a firing head.

Each stapling head 18a-18j of this exemplary embodiment 10 is disposed for movement from an open position about the front end 14 of the anvil 12 to a closed position closer to the front end 14 of the anvil 12. The stapling heads 18a-18j move as a group; they do not move individually between open and closed positions in this embodiment. Other embodiments may vary. For example, in some embodiments, the stapling heads can fire in sequential order around the circumference of the circular aortic stapler 10. Given the radial disposition of the limbs 16a-16j, the open position of the stapling heads 18a-18j has a greater diameter (or radius) than their closed position. In other words, the stapling heads 18a-18j move from their respective open positions in a wider circular pattern to closed positions in a smaller circular pattern about the front end 14 of the anvil 12. A stapling head 18a-18j may be made of the same material as its respective limb 16a-16j, or may be made of a different material. In this example 10, the stapling heads 18a-18j are of two types, which are described below.

The exemplary embodiment 10 includes a cam 20 that may be actuated by a control 22. The cam 20 may be actuated to cause the stapling heads 18a-18j to move from their respective open positions to their closed positions. Additionally, or alternatively, the control 22 can take a variety of forms and employ a variety of activation techniques, such as a trigger or push button mechanism. These control mechanisms can be beneficial in that they require less dexterity to operate, and are equally convenient to operate regardless of whether a physician is right or left handed.

Depicted in its first or open position in FIG. 3, the cam 20 includes a generally cylindrical element encircling the limb ends opposite the stapling heads 18a-18j and the end of the anvil 12 opposite its front end 14. The generally cylindrical element may be referred to as a sleeve or a clamp sleeve. The clamp sleeve of the cam 20 may be disposed to move along the lengths of the limbs 16a-16j and anvil 12 towards a closed or second position closer towards the stapling heads 18a-18j of the limbs and the front end 14 of the anvil 12. For ease of description herein, the “cam” is said to move rather than the “clamp sleeve of the cam”. As the cam 20 moves towards its closed position, the stapling heads 18a-18j may be caused to move from their open positions towards their closed positions about the front end 14 of the anvil 12. The cam 20 may be made of stainless steel (and/or any other appropriate material). The size of the cam is variable so long as it achieves its function.

A control 22 is shown in FIG. 3 as positioned at the end of the embodiment 10 opposite to the front end 14 of the anvil 12, but in other embodiments, the control 22 may be otherwise positioned. The control 22 may be referred to as a rotary control, controller, control knob, knob, OR fire and release knob.

In this embodiment 10, the control 22 may be used to cause the exemplary embodiment 10 to assume its closed position, to fire staples against the anvil 12, and to move to its open position. Particularly, actuating the control 22 by turning it clockwise causes the cam 20 to move from its open position towards its closed position closer to the front end 14 of the anvil 12, thereby causing the stapling heads 18a-18j to move from their respective open positions to their closed positions closer about the front end 14 of the anvil 12. Also in this embodiment 10, further actuating the control 22 by continued turning clockwise causes the stapling heads 18a-18j to fire their respective staples against the side of the anvil 12. The control 22 additionally may be used to return or to move the exemplary embodiment 10 to its open position by causing movement of the cam 20 away from the front end 14 of the anvil 12 so that the stapling heads 18a-18j may move from their small circular pattern about the front end of the anvil 12 to their respective wider circular pattern further from the front end 14 of the anvil 12.

As noted above, the exemplary circular aortic circular aortic stapler 10 may be used to connect a tubular graft to an end of a dissected aorta. A tubular graft may be made of Dacron® in whole. Other embodiments may have tubular grafts of other materials in whole or in part. A tubular graft also may be referred to as a graft, tube, or sleeve.

The following paragraphs provide an overview of how a tubular graft may be positioned on the circular aortic stapler 10 prior to moving the circular aortic stapler 10 with tubular graft for connection to the end of the aorta.

FIG. 3 illustrates the central cylindrical anvil 12 of the exemplary circular aortic stapler 10. The anvil 12 includes an optional feature, e.g., a contour, to aid in holding a tubular graft in place around the anvil 12. In particular, the anvil 12 includes a groove 24 around a circumference of the anvil 12.

In this example 10, the circumferential groove 24 is generally perpendicular to the longitudinal axis of the anvil 12. The groove 24 is generally “u” shaped in cross-section. The groove 24 has a depth to accommodate the diameter of a tie that may be used with the groove 24 to aid in holding the tubular graft in place on the anvil. Alternatively, the groove 24 may have more or less depth than the diameter of a tie that may be used coordinately with the groove 24, may be of a different shape in cross section, and/or may be positioned differently so long as the groove 24 at least partially carries out its function of aiding the tubular graft to stay in place on the anvil 12. Other embodiments of the disclosed subject matter may forgo the use of a feature such as the groove 24 and/or ties.

In the exemplary embodiment 10 shown in FIG. 3, the groove 24 is positioned generally at a distance latitudinally away from the front end 14 of the anvil 12 so as not to interfere or be aligned with the stapling connection made between the tubular graft and the end of the dissected aorta. The groove 24 may be positioned on the anvil so the groove 24 is not covered by the part of the end of the dissected aorta that is positioned over a section of the tubular graft covering the anvil 12.

FIG. 4 shows the same exemplary embodiment 10 as in FIG. 3, but the entire length of a tubular graft 26 is now sleeve-fitted over the anvil 12. Particularly, the tubular graft 26 may be fitted over the anvil by inserting the front end 14 of the anvil 12 into an end of the tubular graft 26 and drawing the tubular graft 26 along the length of the anvil 12 until substantially the entire length of the tubular graft 26 covers the anvil 12. A front end of the tubular graft 26 may align with the front end 14 of the anvil 12, but that does not necessarily have to be the case.

The fit between the tubular graft 26 and the anvil 12 is generally snug in this embodiment 10. In other words, the diameter of the cylindrical anvil 12 may be just slightly less than the diameter of the tubular graft 26. The snug fit may be enough to hold the tubular graft 26 in place around the anvil 12. The exemplary embodiment 10, however, as explained above, includes a feature to help hold the graft 26 in place around the anvil 12. The feature includes the circumferential groove 24 on the anvil 12 shown in FIG. 3.

The groove 24 around the anvil 12 of the exemplary embodiment 10 (shown in FIG. 3) may be used to aid in keeping the tubular graft 26 in place around the anvil 12. As shown in FIG. 4, a tie 28 may be used to tie the tubular graft 26 to the anvil 12. In particular, the tie 28 is tied around the tubular graft 26 and anvil 12 at a position generally above or corresponding to the groove 24 on the anvil 12. The tie 28 may be tied tight enough so the tie 28 when fixed does not add much if any diameter to the combination of the anvil 12 with groove 24, tubular graft 26, and tie 28. To put it another way, the secured tie 28 may lie along with the tied portion of the tubular graft 26 generally in the circumferential groove 24 around the anvil 12. The tie 28 may be fixed in any manner appropriate and that may allow for its easy release so the tubular graft 26 may be pulled away from its position around the anvil 12 of the circular aortic stapler 10.

The tie 28 in the embodiment 10 may be made of suture string, thread, or other material, and may be hand tied. Alternatives to the tie 28 and its characteristics are possible, e.g., a band. In addition, an embodiment of the disclosed subject matter may make use of a tie 28 (or an alternative) as an aid to holding a tubular graft in place on an anvil of an circular aortic stapler, but that embodiment may lack the groove 24 shown in FIG. 3 (or have an alternative feature).

With the tubular graft 26 attached to the anvil 12, the exemplary circular aortic stapler 10 is ready to be moved into the operative field so the graft 26 may be connected to an end of the dissected aorta. In particular, the exemplary circular aortic stapler 10 is positioned so part of the anvil 12 with the tubular graft 26 is inserted into the end of the dissected aorta. The insertion of part of the anvil 12 with the tubular graft 26 causes an overlap between part of the tubular graft 26 and a portion of the dissected end of the aorta.

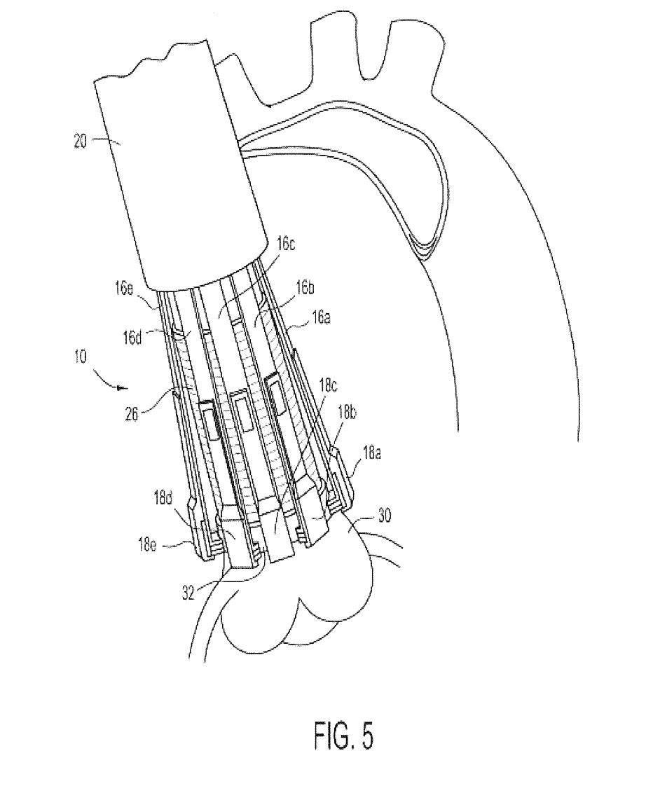

FIG. 5 illustrates part of the same embodiment 10 of FIGS. 3 and 4, but FIG. 5 shows the exemplary circular aortic stapler 10 as it may be initially positioned in the operative field with respect to an end of a dissected aorta 30 to which the tubular graft 26 sleeved over the anvil 12 is to be connected in a leak-proof manner.

Prior to the positioning shown in FIG. 5 of the exemplary circular aortic stapler 10 with sleeved tubular graft 26 with respect to the end of the dissected aorta 30, a collar of graft or graft collar may be positioned around the end of the dissected aorta 30. The graft collar 32 may be used to reinforce the end of the dissected aorta 30. The graft collar 32 may be made of Dacron®. Other embodiments may be made in whole or in part of other materials. The graft collar 32 also may be referred to as a reinforcement ring.

Referring again to FIG. 5, the positioning of the exemplary circular aortic stapler 10 with sleeved tubular graft 26 is described with respect to the end of the dissected aorta having the encircling graft collar 32. In particular, the exemplary circular aortic stapler 10 with sleeved tubular graft 26 is moved into the operative field so the front end of the anvil 12 with tubular graft 26 is inserted into the end of the dissected aorta 30 with graft collar 32. Specifically, the end of the aorta 30 with graft collar 32 overlaps the part of the tubular graft 26 covering the end of the anvil 12 inserted into the end of the dissected aorta 30. The overlap is positioned between the stapling heads 18a-18j (stapling heads 18a-18e are shown) of the limbs 16a-16j (limbs 16a-16e are shown) of the exemplary circular aortic stapler 10 and the end of the anvil 12 inserted into the end of the dissected aorta 30.

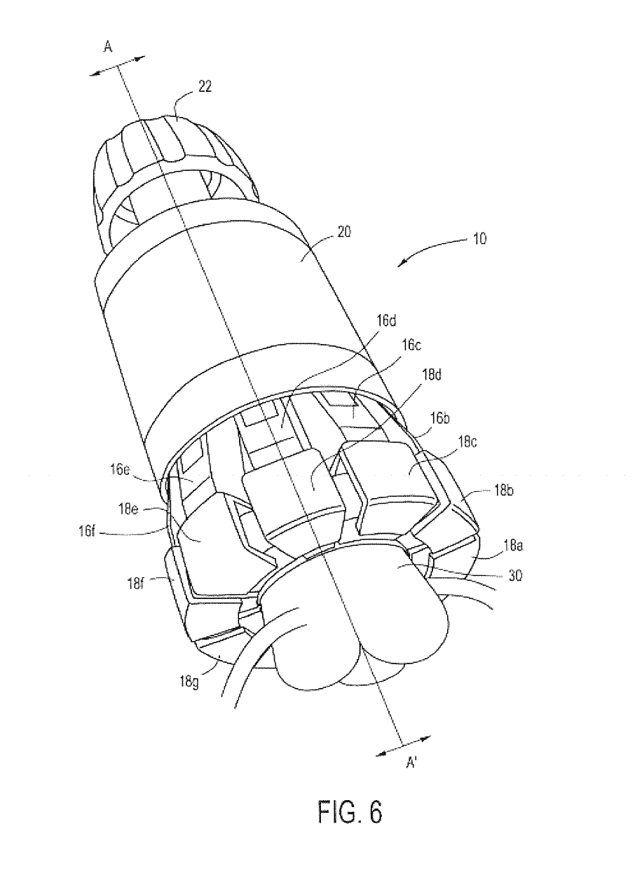

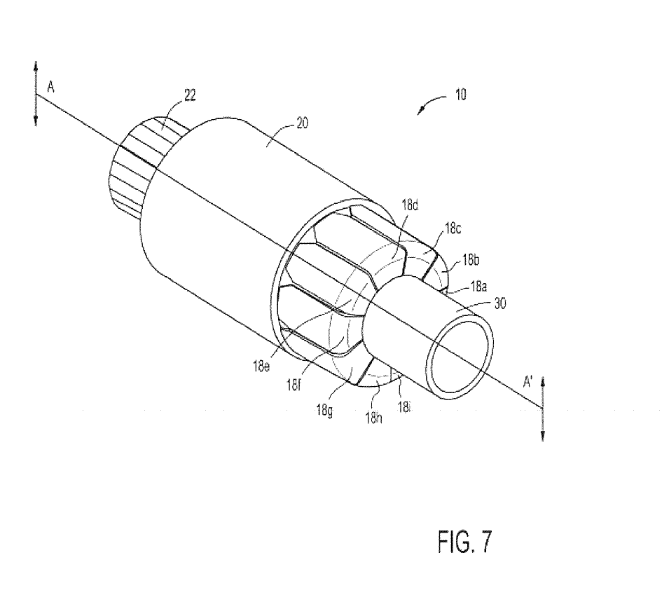

Once the exemplary circular aortic stapler 10 and the end of the dissected aorta 30 with graft collar 32 are positioned so that a portion of the end of the dissected aorta 30 with graft collar 32 lies below the circular aortic stapler's 10 stapling heads 18a-18j (stapling heads 18a-18e are shown), but above the end of the tubular graft 26 covering the anvil 12 inserted into the end of the dissected aorta 30, the exemplary circular aortic stapler 10 may be moved from its open position (as shown in FIG. 5) to a closed position (as shown in FIG. 6) and then to a firing position (as shown in FIG. 7). Some embodiments may omit the closed position, instead moving from an open position to a firing position. Other embodiments may have more positions than the three above-listed positions. In some embodiments, the closed position may be the same as the firing position, but a specific actuation may be necessary to fire the staples rather than an automatic firing once the circular aortic stapler moves into that position.

In its open position (as shown in FIG. 5), the cam 20 is furthest from the front end 14 of the anvil 12 (not shown in FIG. 5) of the exemplary circular aortic stapler 10. To reach its closed position (as shown in FIG. 6), the cam 20 slides along the lengths of the limbs 16a-16j in a longitudinal direction towards the front end 14 of the anvil 12. As the cam 20 slides towards the front end 14 of the anvil, the cam 20 acts on the limbs 16a-16j so the stapling heads 18a-18j move closer towards each other and the front end 14 of the anvil 12. The action of the cam 20 on the limbs 16a-16j may be a lever action. In their closed position, the stapling heads 18a-18j have little if any space between them as may be seen in FIG. 6.

From its closed position as shown in FIG. 6, the exemplary circular aortic stapler 10 may be moved to its open position as shown in FIG. 5 or to its firing position as shown in FIG. 7. Other embodiments may provide other positions so those embodiments may provide other options.

In this embodiment, the circular aortic stapler 10 may be moved to its open position by turning the control 22 in a counterclockwise direction to cause the cam 20 to slide back the way it came and away from the front end 14 of the anvil 12. In particular, the cam 20 may be provided with spring rods to provide reverse tension on the limbs (also referred to as the firing sleeve arms) as the clamp sleeve of the cam is retracted after firing.

Also in this embodiment, the circular aortic stapler 10 may be moved into its firing position by further turning the control 22 in the clockwise direction. Turning the control 22 in such a way causes the cam 20 to interact with the limbs 16a-16j so they carry out a stapling function. Particularly, turning the control 22 further in the clockwise direction causes the cam 20 to continue its sliding movement from its closed position further towards the front end 14 of the anvil 12. The cam's continued movement towards the front end 14 causes the stapling heads 18a-18j of the limbs 16a-16j to move into firing position as shown in FIG. 7, and to fire their respective staples.

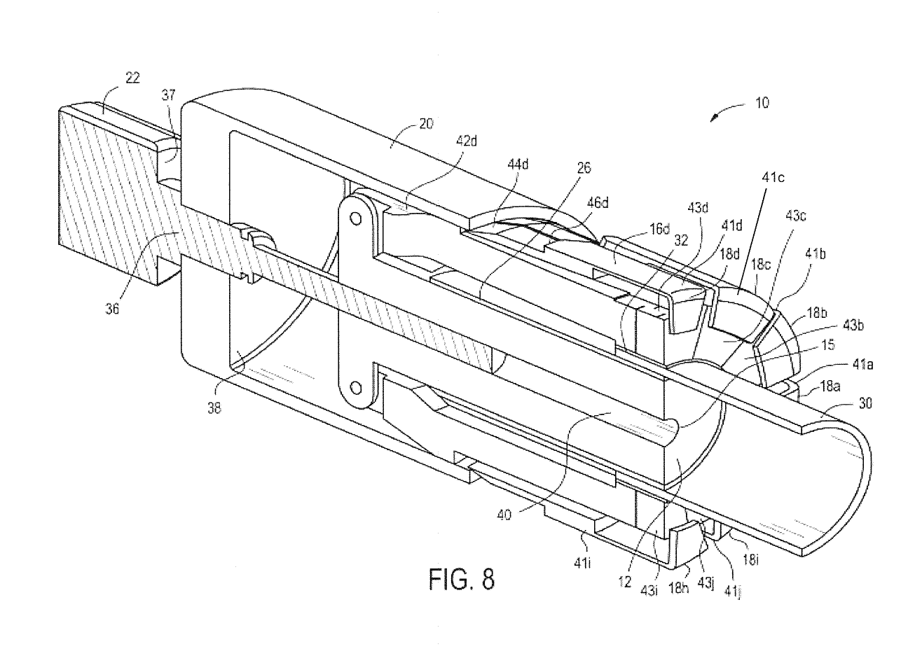

FIG. 8 is a cut-away diagram showing the interior of the exemplary circular aortic stapler 10 for use in explaining the motion of the cam 20 with respect to other elements The cut-away diagram is taken along lines A-A′ marked in FIGS. 6 and 7. The cut-away diagram illustrates the exemplary embodiment 10 in its closed position.

In particular, FIG. 8 shows that a control screw 36 protrudes from the longitudinal end 37 of the control 22 closest to the cam 20. The control screw 36 passes through a central hole in the side face 38 of the cam 20. The side face 38 of the cam 20 is facing the side 37 of the control 22. The side face 38 is generally perpendicular to the longitudinal axis of the cam 20. After passing through the side face 38 of the cam 20, the control screw 36 fits into a central shaft 40 of the anvil 12. The control screw 36 is connected to the central shaft 40 by a slip joint which allows the control screw 36 to rotate and force motion of the cam without turning the anvil 12. When the control 22 is turned, the control screw 36 turns and causes the cam 20 to move either towards or away from the front end 14 of the anvil 12.

FIG. 8 also shows the movement of the cam 20 on the respective tops of the limbs 16a-16j and the spring angle tension therebetween. Segments of the respective tops of the limbs 16a-16j come into contact with the underside of the generally cylindrical element (clamp sleeve) of the cam 20.

In particular, each of the limbs 16a-16j includes an aligned two-part firing mechanism: (1) a firing pin head 41a-41j (41e-41h not shown in FIG. 8) above (2) a part of a staple firing chamber 43a-43j (43a, 43e-43h not shown in FIG. 8). Such a firing mechanism is well known to those familiar with stapler design. Each of the firing pin heads is shorter in length than its corresponding staple firing chamber. In this embodiment, each firing pin head 41a-41j is positioned above and along the front two-thirds (approximate) of its corresponding staple firing chamber 43a-43j.

As the cam 20 moves the circular aortic stapler 10 from its open position to its closed position, the cam 20 comes into direct contact due to spring angle tension first with the tops of the staple firing chambers 43a-43j and then the tops of the firing pin heads 41a-41j of the limbs 16a-16j. As the cam 20 moves along the length of the circular aortic stapler 10, the cam 20 first encounters those parts of the staple firing chambers 43a-43j that are not topped by the firing pin heads 41a-41j. The tops of these initial sections of the staple firing chambers 43a-43j each include a bevel (also referred to as a ramp) that rises towards the front end 14 of the circular aortic stapler 10. The cam 20 causes each of the limbs 16a-16j to move from their open positions to their closed positions when the cam “climbs” the bevels of the staple firing chambers 43a-43j of the limbs 16a-16j.

FIG. 8 depicts the circular aortic stapler 10 in its closed position. To move the circular aortic stapler 10 into firing position, the control 22 is turned further in the clockwise direction. The turning of the control 22 causes the cam 20 to continue its movement along the length of the circular aortic stapler 10, and in particular, along the tops of the firing pin heads 41a-41j of the limbs 16a-16j. The top of each of the firing pin heads 41a-41j has a lower or back section that begins with a bevel (also referred to as a ramp) rising towards the front end 14 of the circular aortic stapler 10. The cam 20 “climbs” the bevels of the firing pin heads 41a-41j towards the front end 14 of the circular aortic stapler 10. The movement of the cam 20 on the firing pin heads 41a-41j forces each firing pin head 41a-41j into its corresponding staple firing chamber 43a-43j. As the cam 20 advances along the circular aortic stapler 10 towards its closure, the cam 20 is in constant contact due to spring angle tension with the firing pin heads 41a-41j of the limbs 16a-16j. The cam 20 applies greater pressure as it moves forward along the tops of the firing pin heads 41a-41j due to the bevels decreasing the distance between the cam and the respective limbs 16a-16j at a greater rate than the forward advancement of the cam 20.

As noted, the top of each limb 16a-16j includes a series of two bevels or ramps. In FIG. 8, only the two ramps 42d, 44d of limb 16d are clearly visible. The first ramp 42d is located generally near the base of the staple firing chamber 43d of limb 16d (i.e., the end of the limb 16d opposite to its staple firing chamber 18d). The first ramp 42d begins near the end of the limb 16d and rises slightly until the ramp 42d ends in a generally perpendicular falling off of the ramp 42d. The second ramp 44d follows the first ramp 42d in series along the top of the limb 16d, but on the firing pin head 41d. Particularly, the second ramp 44d begins where the first ramp 42d falls off and rises slightly before the second ramp 44d levels off to the horizontal for a short distance that ends in a stop that marks the beginning of the staple firing chamber 18d of the limb 16d.

The movement of the cam 20 along the length of the limb 16d towards the front end 14 of the anvil is now described. In the open position of the exemplary circular aortic stapler 10, the cam 20 is generally positioned around the base of the limbs 16a-16j. The turning of the control 22 causes the control screw 36 to rotate thereby moving the cam 20 towards the front end 14 of the anvil 12. As the cam 20 moves along the limb 16d (and the other limbs), the cam 20 encounters the first ramp 42d (as well as the other ramps), slides along it, and exerts downward pressure as the cam 20 moves along. The downward pressure causes the staple firing chamber 18d of the limb 16d to move from its open position about the front end 14 of the anvil 12 to a closer position about the front end 14 that is referred to as the “closed position”. FIG. 8 specifically depicts the exemplary circular aortic stapler 10 in its closed position.

To move the exemplary circular aortic stapler 10 back to its open position from the closed position depicted in FIG. 8, the user may turn the control 22 in the opposite direction. In response, the control screw 36 turns in a direction opposite to its previous turning, and the cam 20 is caused to move away from the front end 14 of the anvil 12. Particularly, the cam 20 moves backwards down the first ramp 42d (as well as the other ramps), and in moving off the ramp 42d, the cam 20 releases the pressure on the limb 16d (and the other limbs). The stapling arm 18d is released to return to its open position.

From its closed position depicted in FIG. 8, the exemplary circular aortic stapler 10 may be moved to a firing position that causes the staples to be fired from the stapling heads 18a-18j. As noted, the cam 20 has been moved up the first ramp 42d of the limb 16d until the cam 20 encounters the perpendicular falling away of the first ramp 42d so as to attain the closed position for the exemplary circular aortic stapler 10. To move the limbs 16a-16j into firing position, the user continues to turn the control 22 in the direction that was used for moving the limbs 16a-16j into the closed position. In response, the control screw 36 turns and causes the cam 20 to continue its movement towards the front end 14 of the anvil 12. In particular, the cam 20 encounters the second ramp 44d on the limb 16d and exerts pressure on the limb 16d. The cam 20 continues its movement and pressure as the control 22 is turned and until the cam encounters a stop 46d at the end of the second ramp 44d on the limb 16d. The stop 46d also marks the beginning of the staple firing head 18d of the limb 16d. As a result of the exerted pressure of the cam 20 on the limbs 16a-16j, the staple firing heads 18a-18j are caused to fire their respective staples.

In this embodiment 10, the firing of the staples may be characterized as a slow deformation of the staples as the cam 20 moves forward along the length of the circular aortic stapler 10 towards its front end 14. As the cam 20 moves forward, more pressure is exerted so the staples are compressed or formed between the anvil and the stapling heads 18a-18j.

An embodiment of the disclosed subject matter may include a feature particular to the control 22 so as to avoid overtightening. Such a control may allow only a certain amount of pressure when rotating to the closed position before the control then locks in that closed position until the direction of rotation is reversed. This is a feature that also may be found in some torque wrenches and screw guns to avoid overtightening.

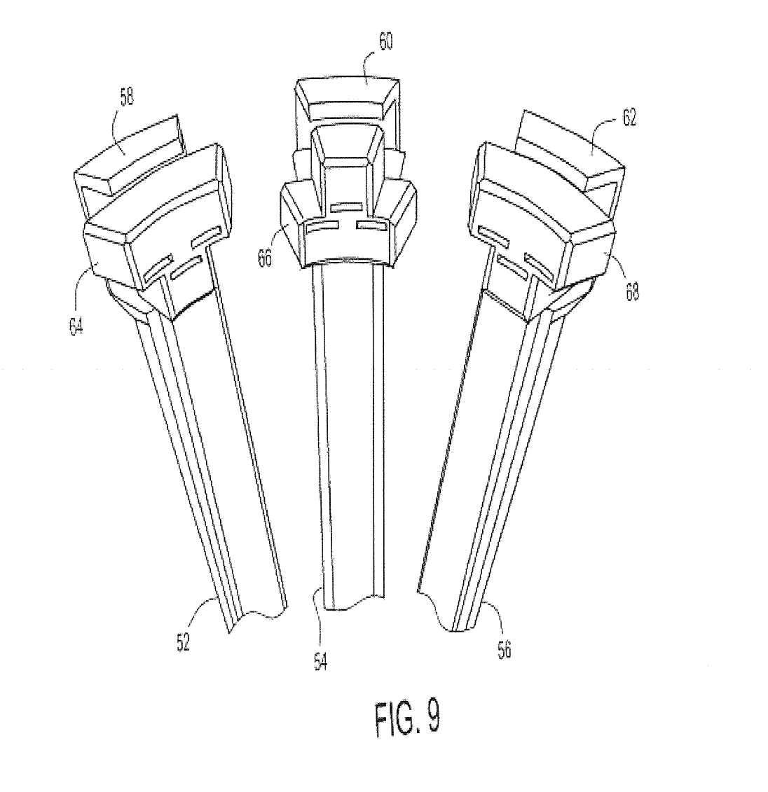

Additional information about features of the limbs that may be used with the exemplary embodiment 10 (as well as other embodiments) is now provided by reference to FIGS. 9, 10 and 11.

FIG. 9 illustrates three exemplary limbs 52, 54 and 56 as may be used with the exemplary embodiment 10 or other embodiments. Each limb 52, 54, 56 includes a respective firing pin head 58, 60, 62. Each limb 52, 54, 56 is generally the same in terms of size, structure, functionality except for the pattern in which each limb 52, 54, 56 fires its staples and the staple firing chambers 64, 66, 68 that facilitate such patterned stapling. In this embodiment, three staples in two rows are fired from each limb 52, 54, 56, but in one of two patterns. The first pattern is two spaced-apart staples in the top row and one staple in the bottom row. Limbs 52, 56 fire staples in the first pattern. The second pattern is one staple in the top row and two spaced-apart staples in the bottom row. Limb 54 fires staples in the second pattern.

In both patterns, the single staple is wider than the space between the staples in the other row. The result of these two firing patterns is that the limbs of the exemplary embodiment fire staples into two circumferential generally parallel but staggered rows. The pattern of the staples in the two rows may be referred to as “interleaved”. Advantageously, the staggered rows of staples makes for a leak proof connection. The staples are all of the same size in this embodiment 10, but do not necessarily have to be, so long as the leak proof connection is attained.

The exemplary embodiment 10 facilitates the use of the two patterns of three staples by including staple firing chambers where a staple firing chamber corresponds to a limb's firing pattern. For example, limbs 52, 56 include respectively staple firing chambers 64, 68. Each staple firing chamber 64, 68 is a generally T-shaped element that allows room for the T-shaped first pattern of staples with two on top and one on the bottom. Limb 54, on the other hand, has an “inverted” T-shaped staple firing chamber 66 as an element. Inverted T-shaped staple firing chamber 66 allows room for the inverted T-shaped second pattern of staples with one on top and two on the bottom.

In the exemplary circular aortic stapler 10, the T-shaped staple firing chambers on the limbs alternate with the inverted T-shaped staple firing chambers. With an even number of limbs, the result is the limbs fire staples into the two circumferential generally parallel but staggered rows.

In the exemplary embodiment 10, the staple firing chambers of the limbs are configured to “mate” so the limbs 16a-16j in the closed and firing positions come together in a substantially closed circle around the front end 14 of the anvil 12. There is not much space between the staple firing chambers of the limbs when they are in their closed and firing positions.

FIG. 9 is used to explain how the staple firing chambers 64, 66, 68 may be made to match up. Staple firing chamber 64 is T-shaped. To the right of staple firing chamber 64, staple firing chamber 66 has an inverted T-shape. The right side of staple firing chamber 64 matches or fits together with the left side of staple firing chamber 66. Specifically, the right arm in the “T” of staple firing chamber 64 fits above the left arm of the inverted “T” of staple firing chamber 66 when their respective limbs are in the closed and firing positions. Similarly, the right arm of the inverted “T” of staple firing chamber 66 fits below the left arm of the “T” of staple firing chamber 68, which is to the right of staple firing chamber 66, when their respective limbs are in the closed and firing positions. Advantageously, it is relatively easy for the staple firing chambers 64, 66, 68 to move out of their matched positions when their respective limbs move to an open position.

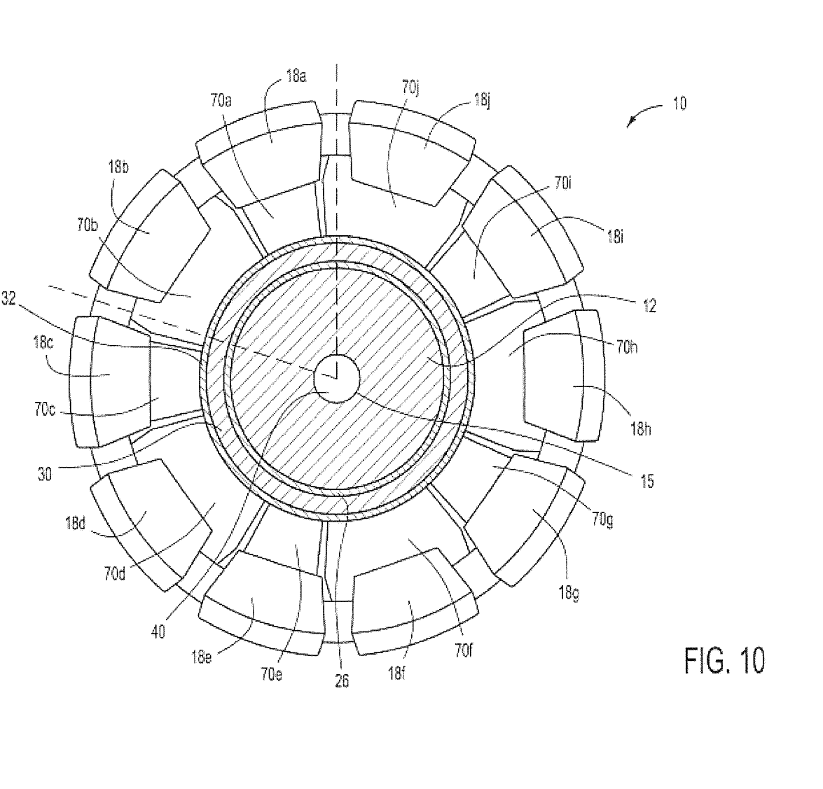

FIG. 10 provides a view of how the staple firing chamber of the limbs 16a-16j in the exemplary embodiment 10 match up to form a circle around the front end 14 of the anvil 12. FIG. 10 is a radial cross section of the exemplary embodiment 10. The center circle represents the core 40 in the anvil 12 for receiving the control screw 36 (although the control screw 36 may not extend all along the length of the core 40). Around the core 40, the front end 14 of the anvil 12 is depicted. In this embodiment 10, the anvil 12 has an outer diameter of about 27.5 mm.

A tubular graft 26 is sleeved about the anvil 12. In this embodiment 10, the cylindrical wall of the tubular graft 26 has a thickness of about 1.0 mm. When the tubular graft 26 is compressed as a result of the stapling connection to the end of the aorta 30 and the graft collar 32, the compressed tubular graft has a thickness of about 0.75 mm.

The circle 30 around the graft 26 represents the end of the dissected aorta. The end of the dissected aorta in this example has a thickness of about 2.0 mm with a compression rate of about 30% or 1.4 mm when staple connected using the exemplary embodiment 10. The outermost circle 32 represents the graft collar that may be placed around the end of the dissected aorta 30 prior to connection of the tubular graft 26 and the aorta 30. The graft collar 32 has a thickness of 1.0 mm prior to compression, but about 0.75 mm after.

The stapling heads 18a-18j of the limbs 16a-16j of the exemplary embodiment 10 are shown in their closed positions encircling the elements listed in the previous paragraph. There are ten (10) stapling heads 18a-18j in this embodiment 10. Other embodiments may have more or fewer. Five (5) of the stapling heads 18b, 18d, 18f, 18h and 18j have T-shaped staple firing chambers 70b, 70d, 70f, 70h, and 70j. The other five (5) of the stapling heads 18a, 18c, 18e 18g, and 18i have inverted T-shaped staple firing chambers 70a, 70c, 70e, 70g, and 70i. The left arms of the T-shaped staple firing chambers 70b, 70d, 70f, 70h, and 70j fit over the respective right arms of the inverted T-shaped staple firing chambers 70a, 70c, 70e, 70g, and 70i. The right arms of the T-shaped staple firing chambers 70b, 70d, 70f, 70h, and 70j fit over the respective left arms of the inverted T-shaped staple firing chambers 70c, 70e, 70g, 70i, and 70a. As noted previously, the fit between the stapling heads 18a-18j is relatively close with hardly if any space between.

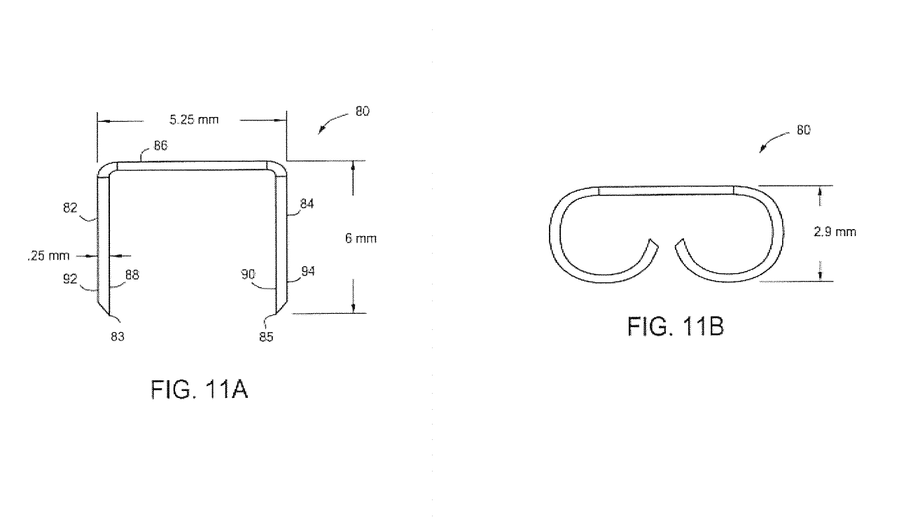

FIGS. 11A and 11B illustrate a staple 80 that may be used with the exemplary embodiment 10. Other connection means and differently sized or shaped staples may be used in other embodiments. FIG. 11A shows the staple 80 as it may be loaded into a staple firing chamber 70a-70j of a limb 16a-16j. Prior to stapling, as shown in FIG. 11A, the staple 80 may be referred to as “unformed”; after stapling, as shown in FIG. 11B, the staple 80 may be referred to as “formed”.

The unformed staple 80 is generally U-shaped having two generally parallel sides 82, 84 and a top 86 generally perpendicular to the sides 82, 84. There are ends 83, 85 of the sides 82, 84 of the staple 80 opposite to the top 86. The ends 83, 85 also may be referred to as “prongs” or “tips”. These ends 83, 85 are cut at an angle of about 45 degrees such that the inside sides 88, 90 of the sides 82, 84 of the staple 80 are slightly longer than the outside sides 92, 94. There may be advantages to the ends 83, 85 of the staple 80 having angled cut ends. When the staple 80 is “stapled”, it is forced through the materials it is connecting. The angled ends 83, 85 may work their way through the materials more easily than blunt ends. Also, when the staple 80 is “stapled”, it encounters the anvil 12 after the staple has been forced through the materials. The relatively forceful encounter with the anvil 12 may cause the sides 82, 84 of the staple 80 to change shape. The force may distend the sides 82, 84 into a semi-circular shape as may be seen in FIG. 11B. In particular, the sides 82, 84 may distend in a curved manner outwards from their perpendicular relationship to the top 86 of the staple 80. The formed staple 80 therefore may be wider than the unformed staple. In addition, the ends 83, 85 of the sides 82, 84 staple 80 bend inward, towards each other and then towards the inside top of the staple as shown in FIG. 11B. The bending of the staple sides, and particularly the ends 83, 85 may aid in the security of the connection of the materials that are stapled.

Exemplary staple 80 is made all of one material (titanium), but does not necessarily have to be. The exemplary staple 80 is sized as follows, but as noted, other sizes of staples may be used in other embodiments. In the unformed staple 80, the sides 82, 84 are 6.0 mm in length. The top 86 of the staple 80 is 5.25 mm wide. The overall thickness of the staple 80 is 0.25 mm. When the staple is formed, the width of the staple 80 is slightly larger than its original size and its length is 2.9 mm. The size of the staple 80 may correspond to the elements the staple 80 connects. In this example, the formed staple 80 is long enough (2.9 mm) to connect the tubular graft 26, the end of the aorta 30, and the graft collar 32, which in their compressed state have a total diameter of about 2.9 mm.

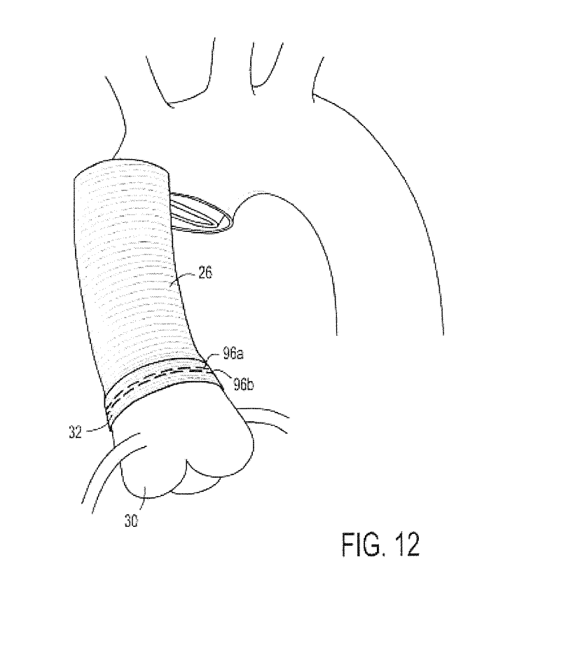

FIG. 12 illustrates an exemplary result of use of the exemplary circular aortic stapler 10, which, itself, is not shown, presumably because it has been removed from the operative field. The use of the exemplary circular aortic stapler 10 has resulted in one end of the dissected aorta 30 having been connected via two circumferential rows of staples 96a, 96b between the graft collar 32 on the outside of the end of the dissected aorta 30 and the tubular graft 26 on the inside. The two circumferential rows of staples 96a, 96b are spaced about 3.0-4.0 mm apart from each other. In each row 96a, 96b there are spaces between staples. The rows 96a, 96b, however, are staggered in their patterns so that a space in either staple row 96a, 96b is “covered” by a staple in the opposite row. In this embodiment, the staples are wider than the space between staples. Thus, the staggered pattern allows for a leak-free connection between the end of the aorta 30 and the tubular graft 26.

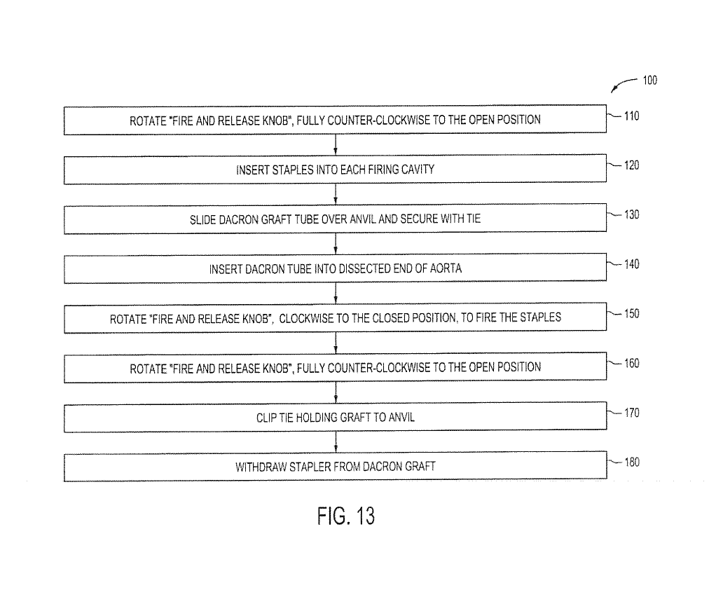

The disclosed subject matter also include methods of anastomosis. An exemplary method 100 is now described with reference to the flow diagram shown in FIG. 13. The exemplary method 100 makes use of the exemplary circular aortic stapler 10 previously described, but other embodiments may be used.

The exemplary method 100 may begin with actions that may be optional depending on the circumstances relating to checking and/or setting up of the circular aortic stapler 10. For example, to begin the method, the user may check whether the circular aortic stapler is in its “open” position (i.e., limbs away from the anvil). In action 110, if the circular aortic stapler needs to be “opened”, then the user may actuate the control to cause the exemplary circular aortic stapler to be in its “open” position. In one particular embodiment, the control may be a “fire and release knob” that is turned to a fully counter-clockwise position to accomplish opening of the circular aortic stapler.

As another example of actions that may be optional to the exemplary method 100, the user may check whether the circular aortic stapler 10 needs to be loaded with staples. If staples need to be loaded, then in action 120 the staples may be inserted into the respective firing cavities in the stapling heads. In the exemplary embodiment 10, the staples may be individually hand-loaded into the circular aortic stapler. Other embodiments may automatically load staples, or may make use of circular aortic stapler cartridges, which may be manually or automatically loaded. The order of the optional actions 110, 120 with respect to each other and/or to the other actions of the method 100 may vary from embodiment to embodiment, and should not be considered as restricted to the order presented herein.

The exemplary method 100 includes the action of positioning a tube to fit around a cylindrical anvil of a circular stapler. Specifically, in action 130, a Dacron® graft tube may be slid over the anvil. In the exemplary method 100, most if not all of the tube is sleeved over the anvil of the circular aortic stapler so that a segment of the tube is relatively close to if not substantially next to the front end of the anvil of the circular aortic stapler. Action 130 of this exemplary method also includes securing the tube to the anvil by releasably tying the tube to the anvil. Other embodiments may not require this tying action and/or may secure the tube in other ways.

In action 140 of FIG. 13, the circular aortic stapler is introduced into the operative field, and specifically, the graft tube on the anvil is inserted at least partially into a dissected end of the aorta. After insertion, a segment of the graft tube underlies a part of the dissected end of the aorta to form an overlap between the graft tube segment and the end part of the dissected aorta.

In action 150, the circular aortic stapler is used to make the leak-free connection between the graft tube and the end of the dissected aorta. Particularly, in this embodiment, the control (also referred to as the fire and release knob) is turned in a clockwise direction so that the limbs of the circular aortic stapler move into their closed positions around the overlap. The control then is turned further clockwise to cause the stapling heads to fire the staples into the overlap. In this embodiment, the firing of the staples causes each staple to pierce the overlap and to at least partially close behind it by an inward movement of each leg of the staple when it encounters the anvil. The firing position of the stapling heads may be characterized in this embodiment as a radial distribution about the overlap of the graft tube and the end of the dissected aorta. The staples may be fired so that the result is two parallel rows of staples around the overlap. The rows of staples (and the materials they compress) form a leak-free connection between the graft tube and the dissected end of the aorta.

To remove the circular aortic stapler from the operative field, in action 160 the exemplary method provides for the control of the circular aortic stapler to be rotated in a counter-clockwise direction to open the limbs of the circular aortic stapler. In optional action 170, any tie and/or other means of securing the graft tube to the anvil of the circular aortic stapler is undone or unsecured so the anvil may be withdrawn from the graft tube. In action 180, the circular aortic stapler is removed so the graft tube remains in its leak-free connection to the dissected end of the aorta.

As noted, an exemplary embodiment of the disclosed subject matter may be used in an anastomosis to connect one end of a tubular graft in a leak-free connection with an end of a dissected aorta. The respective other ends of the tubular graft and the dissected aorta may also need to be connected in a leak-free manner, and such connection may be accomplished in any appropriate manner and/or with any appropriate device. For example, the ends not connected according to the disclosed subject matter may be connected by suturing or any other method of bonding. As will be described in detail herein below, an embodiment of the disclosed subject matter is particularly well-suited to provide a leak-free connection at the end of the tubular graft and dissected aorta in a rapid, reproducible manner, which does not subject the vessel walls to tearing and other trauma that may result from conventional suturing.

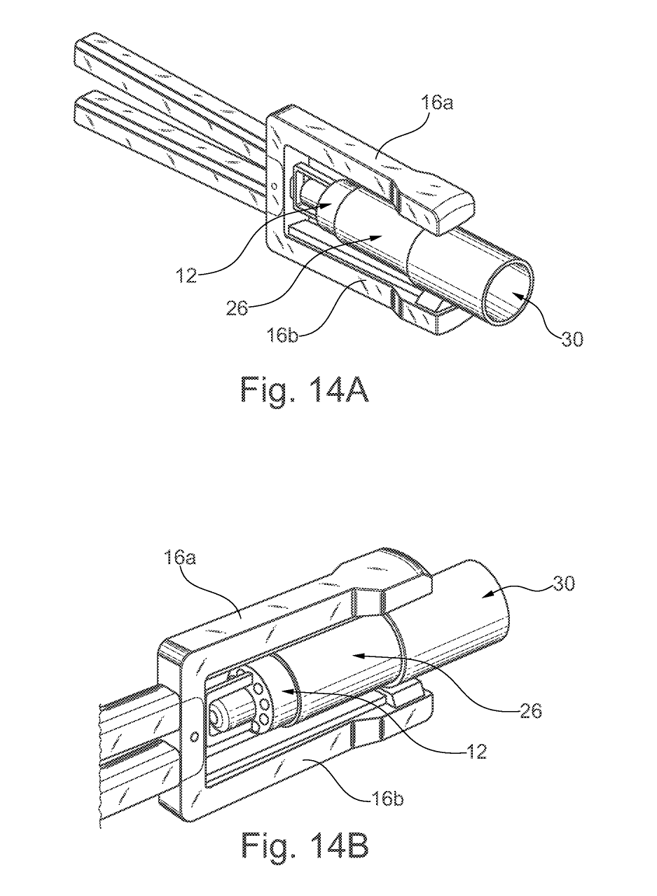

In accordance with another aspect of the disclosed subject matter, a stapler can be configured with at least one pair of stapling limbs which are coupled to each other to move in unison. In the exemplary embodiment illustrated in FIGS. 14A-B, the stapling limbs 16a, 16b are positioned in a diametrically opposed relationship for providing a connection between graft 26 and vessel 30. This is advantageous, e.g., in that the force imparted on anvil 12 from stapling limb 16a will be countered by the equal and opposing force imparted on anvil 12 from stapling limb 16b.

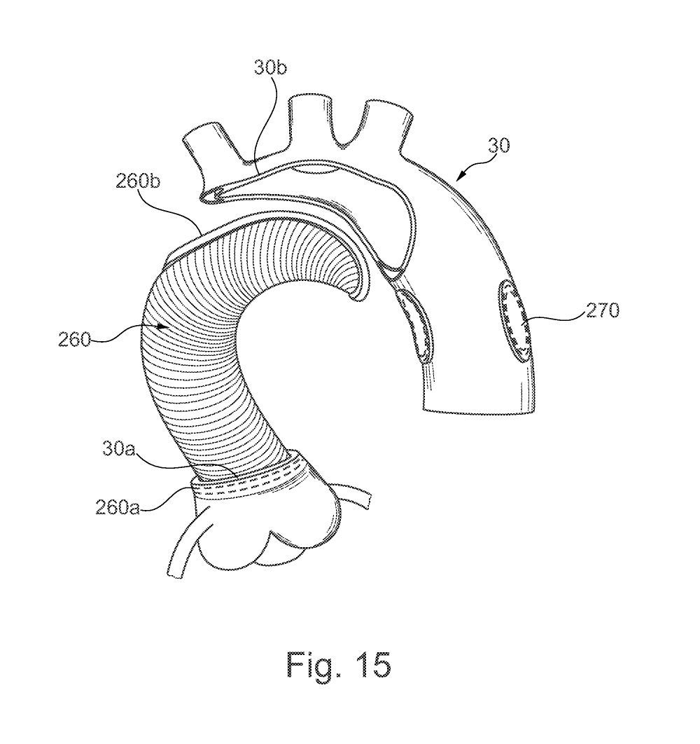

In accordance with another aspect of the disclosed subject matter, a further stapler is provided having stapling limbs arranged side-by-side and which move to engage a base or anvil in substantially the same direction. FIG. 15 illustrates a dissected aorta 30 having both a circular opening 30a (lower portion of illustration) and a non-circular opening 30b (upper portion of illustration). The circular aortic stapler device described above with reference to embodiment 10 can be employed to connect the end 260a of graft 260 to the circular opening 30a of the aorta 30 in a leak-proof manner. In such case, as described above, the graft 260 and the aorta 30 are positioned in a surrounding configuration over the anvil 12 of the circular aortic stapler 10 that provides staples in two circumferential rows 96a, 96b as described above.

In the case of a connection between the end 260b of a graft 260 and non-circular opening 30b of the aorta 30, the provision of a row of staples to make a leak-free connection precludes the placement of the vessel and graft over the anvil of a circular aortic stapler in the manner described hereinabove and/or requires the staples to be applied in a less regular configuration.

An arcuate stapler 210 (described in greater detail below) is advantageous in that it allows for only select portions of the graft to be connected to the aorta in a given stapling function. Therefore, the operator can adjust the positioning of the graft and/or stapler with respect to the aorta to achieve a tailored connection that follows the particular contour of the aorta opening.

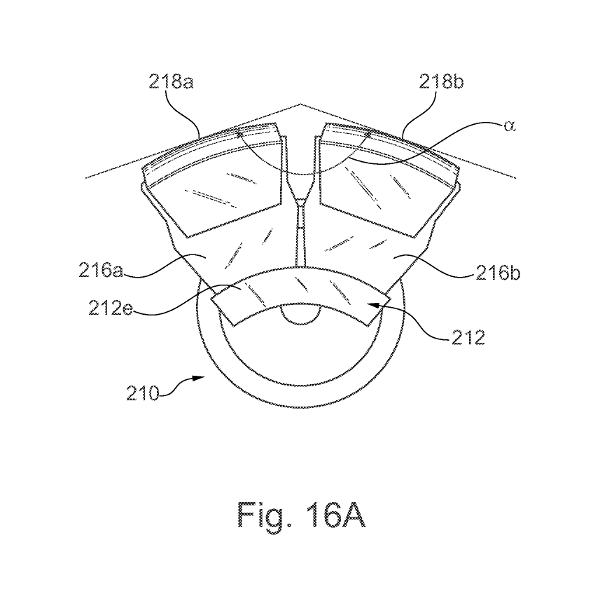

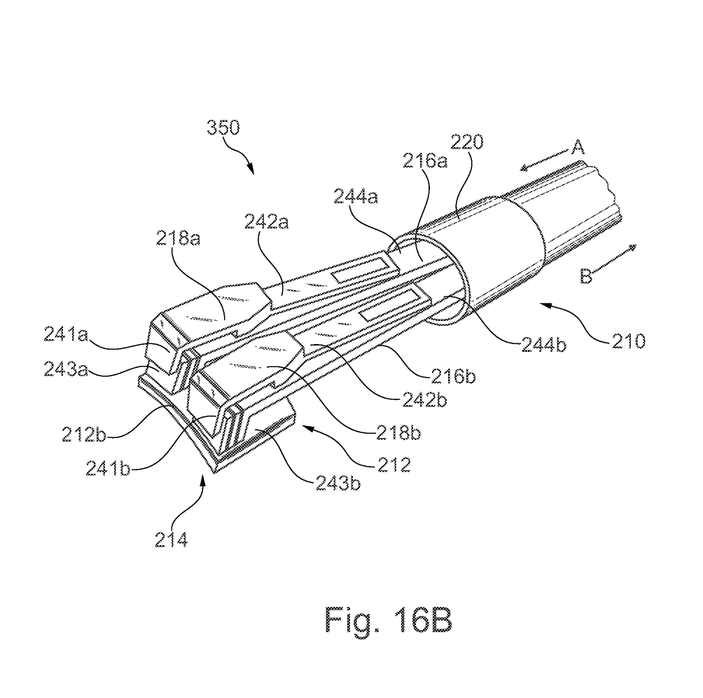

For example, the tops 86 of staples 80 (not shown in FIG. 16A, staples illustrated in FIG. 11A-B) are held by the stapling heads 218a, 218b substantially parallel to one another or at an angle α of about 180° to about 90°. In a particular embodiment of the arcuate stapler, the pair of stapling heads 218a, 218b comprise approximately ⅕ of the full circumference of a circle. The inner contour 212e of the staple base (or anvil) 212 can be substantially flat or convex. An exemplary stapler according to this embodiment can be configured as an arc-shaped, or arcuate, stapler. In the case of an arcuate stapler, the staple tops are maintained at an angle α less than 180°, and the inner contour 212e of the base 212 is arcuate/convex. An arcuate configuration is particularly advantageous for forming leak-tight connections between curved but non-circular (e.g., asymmetrical) vessel openings and/or graft materials. Although the term “arcuate stapler” is used herein, it is understood that the orientation of the stapling heads may be linear (e.g., in which the staple tops are maintained substantially in parallel—angle α of about 180°—and the contour 212e of the base 212 is substantially flat), or in arcuate or angled relationship, as is appropriate to the operative conditions. Moreover, the arcuate stapler is advantageous for providing connections between vessels and graft materials in which only one of the graft sections is freely accessible, or in situations where it is impossible or undesirable to insert a stapler into the vessel and/or graft section.

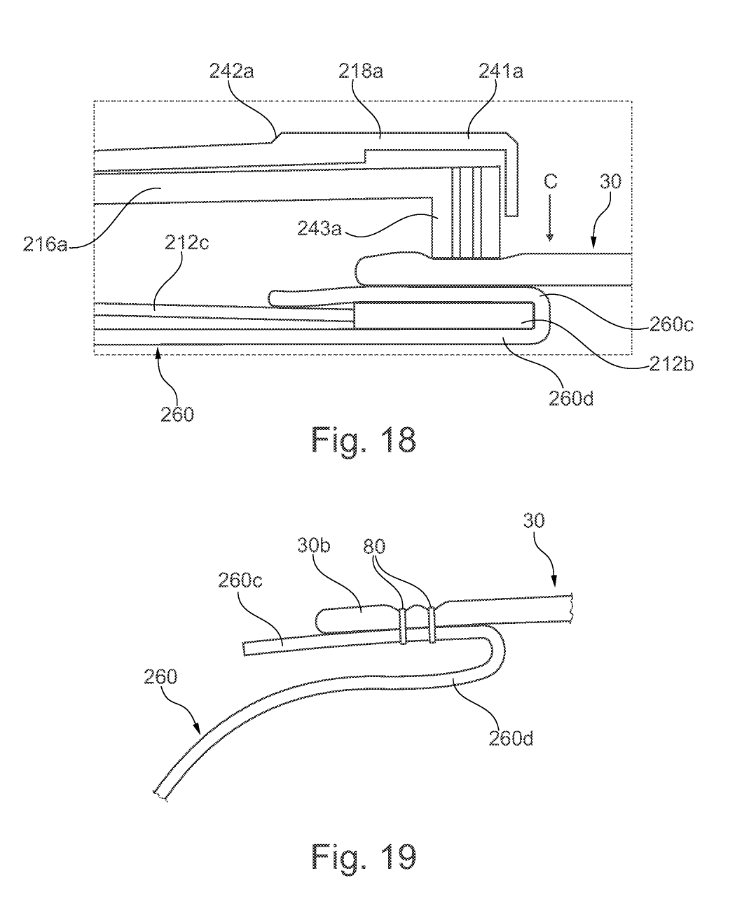

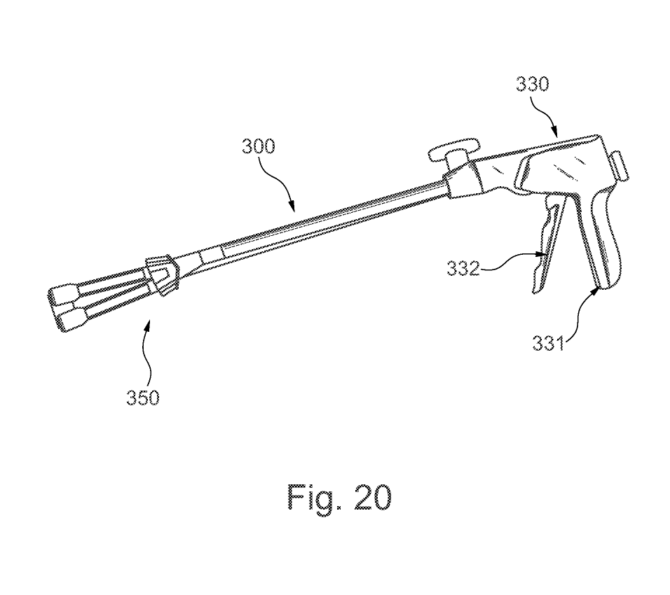

With continued reference to FIG. 16A, base 212 extends along the longitudinal axis of the arcuate stapler 210 and includes an elongate front end portion 212b which corresponds with the location of the stapling operation. (As can be seen in FIG. 18, base 212 includes a front end portion 212b and a support portion 212c which extends longitudinally from the front end of the stapler 210 towards the control (back) end of the device. The end 330 is illustrated in an exemplary embodiment in FIG. 20.) As shown, the front end 212b of the base 212 in some embodiments has an arcuate shape extending in a radial direction around less than 360°. For example, the base may extend for ⅕th of a complete circumference. In some embodiments, the front end portion 212b of the base may substantially flat. Stapling limbs 216a, 216b extend along the length of the base 212 and include stapling heads 218a, 218b at the front end of the device. The base 212 of the arcuate stapler 210 functions as an anvil, against which the staples are driven by the stapling limbs 216a, 216b, discussed below. The base 212 may be made of stainless steel (and/or other appropriate material). The size of the base 212 may vary depending on the size and number of stapling limbs 216a, 216b to be used with the base 212, and also the size of the opening to be spanned by the graft. The size of the graft, in turn, may vary depending on the size of the end of the dissected aorta with which the graft is to be connected. For example, the greater the width of the base 212, the fewer number of stapling operations that would be required to complete the anastomosis.

The stapling limbs 216a, 216b of the exemplary arcuate stapler 210 move between open and closed positions via the control and cam operation of a cam 220, depicted in FIG. 16B. Once the exemplary arcuate stapler 210 and the end of the dissected aorta 30b with graft 260b are positioned (as described herein), the exemplary arcuate stapler 210 may be moved from its open position (as shown in FIG. 16B) to a closed position and then to a firing position. Some embodiments may omit the closed position, instead moving from an open position to a firing position. Other embodiments may have more positions than just the three listed positions. In some embodiments, the closed position may be the same as the firing position, but a specific actuation may be necessary to fire the staples rather than an automatic firing once the arcuate stapler moves into that position.

In its open position (as shown in FIG. 16B), the cam 220 is furthest from the front end 212b of the base 212. To reach its closed position, the cam 220 slides along the lengths of the limbs 216a, 216b in a longitudinal direction (as denoted by arrow A) towards the front end 212b of the base 212, driven by actuation of the pivoting lever 332 towards the grip 331 (as shown in FIG. 20), e.g., against a spring tension, as is known in the art. As the cam 220 slides towards the front end 212b of the base 212, the cam 220 acts on the limbs 216a, 216b so the stapling heads 218a, 218b move closer towards the front end 212b of the base 212. The action of the cam 220 on the limbs 216a, 216b may be a lever action. In some embodiments, the stapling heads 218a, 218b move closer together and closer to the base 212 as they move towards the closed position.

From its closed position, the exemplary arcuate stapler 210 may be moved to its open position or to its firing position. Other embodiments may provide other positions in addition to the three positions discussed above, and those embodiments may provide other options.

In this embodiment, the arcuate stapler 210 may be moved to its open position by releasing the pivoting lever 332 (as described below) to cause the cam 220 to slide back the way it came (as denoted by arrow B) and away from the front end 212b of the base 212. In particular, the cam 220 may be provided with spring rods to provide reverse tension on the limbs (also referred to as the firing sleeve arms) as the clamp sleeve of the cam is retracted after firing the staples.

Also in this embodiment, the arcuate stapler 210 may be moved into its firing position by further actuation of the handle 330. Further advancement of the cam 220 (in the direction of arrow A) causes the cam 220 to interact with the limbs 216a, 216b so they carry out a stapling function. Continued movement of the cam 220 towards the front end 212b causes the stapling heads 218a, 218b of the limbs 216a, 216b to move into firing position and to fire their respective staples.

Each of the limbs 216a, 216b includes an aligned two-part firing mechanism: (1) a firing pin head 241a, 241b positioned above (2) a part of a staple firing chamber 243a,b. The staple firing chambers 243a, 243b can be substantially identical to staple firing chamber 64, 66, and 68 illustrated hereinabove in FIG. 9, and, e.g., be capable of applying two rows of staples with the staples in each row being staggered with respect to the staples of the other row. Each of the firing pin heads is shorter in length than its corresponding staple firing chamber. In this embodiment, each firing pin head 241a, 241b is positioned above and along the front two-thirds (approximate) of its corresponding staple firing chamber 243a,b.

In an exemplary embodiment as shown in FIG. 16B, stapling heads 218a, 218b are disposed on stapling limbs 216a, 216b. For example, stapling heads 218a, 218b can be hinged with respect to stapling limbs 216a, 216b at junctions 244a, 244b. As the cam 220 moves along the length of the arcuate stapler 210, the cam 220 first encounters those parts of the stapling heads 218a, 218b. Thus, the tops of these initial sections of the staple heads 218a, 218b each include a ramp at 244a, 244b that rises towards the front end 214 of the arcuate stapler 210. Engagement of the cam 220 with the ramp 244a, 244b causes each of the limbs 216a, 216b to move from their open positions to their closed positions when the cam 220 “climbs” the ramps 244a, 244b of the heads 218a, 218b.

To move the arcuate stapler 210 from the closed position into the firing position, the lever 332 is further approximated with the grip 331, which causes the cam 220 to continue its movement along the length of the arcuate stapler 210, and in particular, along the tops of the firing pin heads 241a, 241b of the limbs 216a, 216b. The top of each of the firing pin heads 241a, 241b has a lower or back section that begins with a bevel (also referred to as a ramp 242a, 242b) rising towards the front (distal) end 214 of the arcuate stapler 210. The cam 220 “climbs” the bevels of the firing pin heads 241a, 241b towards the front end 214 of the arcuate stapler 210. The movement of the cam 220 on the firing pin heads 241a, 241b forces each of the firing pin heads 241a, 241b into its corresponding staple firing chamber 243a,b. As the cam 220 advances along the arcuate stapler 210 towards its closure, the cam 220 is in constant contact due to spring angle tension with the firing pin heads 241a, 241b of the limbs 216a, 216b. The cam 220 applies greater pressure as it moves forward along the tops of the firing pin heads 241a, 241b due to the bevels decreasing the distance between the cam and the respective limbs 216a, 216b at a greater rate than the forward advancement of the cam 220.

From the closed position, the exemplary arcuate stapler 210 may be moved to a firing position that causes the staples to be fired from the stapling arms 218a, 218b. As noted, the cam 220 has been moved up the first ramps 244a, 244b of the limbs 216a, 216b so as to attain the closed position for the exemplary arcuate stapler 210. To move the limbs 216a, 216b into firing position, the user continues to advance the lever 332 that was used for moving the arms into the closed position. In response, cam 220 continues its movement towards the front end 214 of the base 212. In particular, the cam 220 encounters the second ramps 242a, 242b on the limbs 216a, 216b and exerts pressure on the limbs 216a, 216b. As a result of the exerted pressure of the cam 220 on the limbs 216a, 216b, the stapling heads 218a, 218b are caused to fire their respective staples.

Although FIGS. 16A-B depict two adjacent stapling limbs, an alternative number of stapling limbs can be employed (with a commensurate base 212b) in the stapler embodiment 210, if so desired. The stapling limbs 216a, 216b can be coupled such that they move in tandem between open and closed positions. Alternatively, each stapling limb can be independently moved from between open and closed positions. Similarly, the stapling heads 218a, 218b can be configured to fire staples simultaneously or independently, as so desired.

In some embodiments the stapling limbs may omit the closed position, instead moving from an open position to firing position. Other embodiments may have additional or intermediate positions. In some embodiments, the closed position may be the same as the firing position, but a specific actuation may be necessary to fire the staples rather than an automatic firing once the stapler moves into that position.

The base 212 may be made of stainless steel (and/or other appropriate material of sufficient strength and rigidity to receive and deform the staples). The size of the base may vary depending on the size of the graft to be used with the base. The size of the graft, in turn, may vary depending on the size of the end of the dissected aorta with which the graft is to be connected. Thus, the size of the base may vary depending on the size of the end of the dissected aorta to which the graft is to be connected in a leak-proof manner.

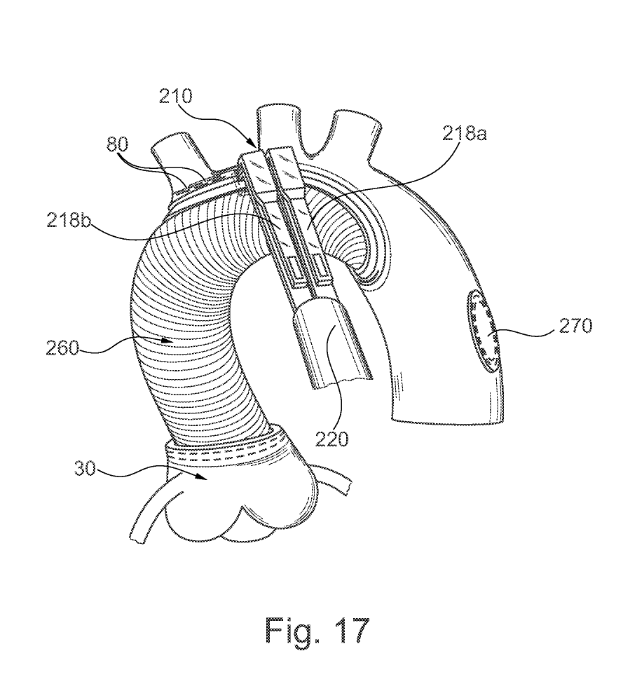

FIGS. 17 and 18 are discussed in conjunction herein. In operation, a graft 260 is positioned on the base 212 of the stapler 210, such that the stapling heads 218a, 218b and stapling limbs 216a, 216b of the stapler are disposed in an open position relative to the base. (Stapling head 218b and stapling limb 216b are not shown in FIG. 18.)

The graft material (typically having an asymmetric or non-circular shaped opening) is folded over itself at the opening 260b to form an external cuff, as shown in FIG. 15. As shown in FIG. 18, the arcuate front end 212b of the base 212 is then inserted within the cuffed portion 260c of the graft 260 such that a layer of the graft material is positioned on the top (or exterior) and bottom (or interior) surfaces of the arcuate front end 212b. The graft 260 receives the front end 212b within the folded portion which allows the front end 212b to be inserted within the opening of the aorta 30 while securely retaining the graft 260 material in a fixed position with respect to the base 212. This is advantageous in that it eliminates the need for any additional equipment, such as filament or ties, to secure the graft to the base.

The base 212, with the graft 260 disposed around the front end 212b, may be positioned inside an end of the dissected aorta 30 so the end of the aorta lies between the splayed limbs 216 of the stapler 210 and the front end 212b. As such, an overlap region is defined having the aorta 30 as the first (or exterior) layer, with the first graft cuff layer 260c underneath, and both the aorta 30 and first graft cuff layer 260c overlying the front end 212b of the base 212, and second graft cuff layer 260d disposed underneath (or interior to) the aorta layer. In such configuration, the aorta 30 and first graft cuff layer 260c are disposed between the stapling limb 216 and the front end 212b. In some procedures, the second graft cuff layer 260d may extend perpendicular to the first graft cuff layer 260c.

Upon activation, the stapling limbs 216a, 216b move from their respective open positions to closed positions relative to each other and to and about the front end 212b of the base (in the direction noted by arrow C). The arcuate stapler 210 may be further activated to cause the stapling heads 218a, 218b to staple the end of the aorta 30 to the first graft cuff layer 260c of the graft 260 at the overlap between the two on the front end 212b of the base 212. The staples pierce through the aorta 30 and first graft layer 260c and deform upon engaging the front end 212b of the base 212, thereby leaving the second graft layer 260d a continuous and unaffected layer. The stapling effects a substantially leak-free connection between the end of the dissected aorta and the graft 260. To remove the stapler 210, its limbs 216 may be caused to open and the base 212 may be slid from the connected aorta and graft without disturbing the leak-free connection therebetween. The arcuate stapler 210 can then be repositioned at a location adjacent to the first stapling location, and within the overlap region, to conduct a second stapling operation. These discrete stapling steps can be repeated as necessary around the circumference of the junction to achieve a leak-tight connection between the aorta and graft along a tailored and asymmetrical pattern.

As shown in FIG. 17, the arcuate stapler 210 can be used in the same manner to provide patches 270 of graft material to replace sections of a vessel.

Accordingly, the arcuate stapler 210 can securely retain and position the graft 260 within the aorta opening without the use of any ties or clips. This serves to streamline the procedure as well as reduce the likelihood of damage or injury to either the graft 260 or the aorta 30. Additionally, the arcuate stapler can be configured and employed for use on handle-controlled and -actuated embodiment 300, as shown in FIG. 20. In some embodiments, apparatus 300 includes an operative portion 350 and a handle or actuating portion 330. The operative portion 350 includes, e.g., base 212, stapling limbs 216, stapling heads 218 and cam 220, as described above, for effecting the stapling action of the graft and vessel. Actuating portion 330 may include a mechanism for remotely actuating the cam 220, such as a grip 331 and pivoting lever 332, as shown in FIG. 20, or a rotating control 22, as shown in FIGS. 3-4 and 6-8.

Although the descriptions herein are made with reference to the connection between a graft and a dissected portion of an aorta or other vessel, it is understood that the apparatus and methods described herein are useful for connecting other elements, such as two vessels, in a leak-proof manner.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. An apparatus for anastomosis, comprising:
a base having a length from a front end and defining a longitudinal axis;
at least two stapling limbs disposed proximate the base, each stapling limb having stapling functionality at its limb end corresponding to the front end of the base;
each stapling limb supporting one or more staples such that staple heads of staples supported by a first stapling limb are maintained at an angle less than 180° with respect to staple heads in an adjoining stapling limb;
each stapling limb end being configured for movement from an open position at a distance from the front end of the base to a closed position closer to the front end of the base;
a control capable of being selectively activated to cause movement of each stapling limb end between the open and closed positions; and
the front end of the base being configured to engage at least a portion of a graft for connection to a vessel,
the base and graft being insertable at least partially into a portion of the vessel with at least a portion of the vessel, graft, and base overlapping each other, and
the control configured to cause each stapling limb in the closed position to fire at least one staple into the overlapping portions of the vessel and graft in a direction perpendicular to the longitudinal axis to make a connection therebetween.

2. The apparatus of claim 1, wherein the front end of the base is configured with an arcuate shape.

3. (canceled)

4. The apparatus of claim 1, wherein the control actuates a cam to cause movement between open and closed positions.

5. The apparatus of claim 1, wherein the front end of the base is received within a folded portion of the graft, the front end of the base having a layer of the graft on opposing first and second surfaces of the base.

6. The apparatus of claim 1, wherein the base and each stapling limb are configured to engage a graft having an asymmetrical end portion.

7. The apparatus of claim 1, wherein the connection comprises a substantially leak-proof connection.

8. The apparatus of claim 1, wherein each stapling limb includes two or more rows of staples.

9. The apparatus of claim 8, wherein staples are fired into the overlapping layers of the vessel and graft forming two or more generally parallel rows, the staples of each row being staggered with respect to the staples of the other row or rows.

10. The apparatus of claim 9, wherein staples from each row of each stapling limb are fired substantially simultaneously.

11. A device for substantially leak-free fastening of at least two elements, comprising:
a base having a fastening end, the fastening end having first and second opposing surfaces and defining a longitudinal axis;
at least one fastening mechanism positioned about the fastening end of the base comprising at least two stapling limbs supporting one or more staples such that staple heads of staples supported by a first stapling limb are maintained at an angle less than 180° with respect to staple heads in an adjoining stapling limb and configured for movement between an open position about the fastening end and a firing position about the fastening end;
wherein the base is configured to receive a length of a first element disposed over the first surface of the base with the fastening mechanism in the open position, and
wherein the fastening end of the base is configured to be received in a portion of a second element to form an overlap between a portion of the first element, the second element and the fastening end of the base with the fastening mechanism in the open position; and
a control configured to activate the at least one fastening mechanism to move from the open position into the firing position about the fastening end to make a substantially leak-free fastening between the first and second elements at the overlap by firing at least one staple into the overlapping portions of the vessel and graft in a direction perpendicular to the longitudinal axis.

12. The device of claim 11, wherein the at least one fastening mechanism is responsive to an opening activation to move away from the firing position to allow for withdrawal of the fastened first and second elements from the base.

13. The device of claim 11, wherein the fastening end of the base is configured with an arcuate shape.

14. The device of claim 11, wherein the base is configured to receive a length of a first element disposed over the first and second opposing surfaces of the base with the fastening mechanism in the open position.

15. A method for anastomosis, comprising:
positioning a graft on a base of an arcuate stapler, the stapler comprising at least two stapling limbs having stapling functionality, each stapling limb supporting one or more staples such that staple heads of staples supported by a first stapling limb are maintained at an angle less than 180° with respect to staple heads in an adjoining stapling limb, the stapling heads of the stapler disposed in an open position relative to the base;
inserting the base into a dissected end of an aorta so that a segment of the graft underlies a portion of the dissected end of the aorta to form an overlap between the graft and the aorta;
moving the stapling heads into a firing position relative to the base within the overlap;
firing staples in two parallel rows within the overlap to form a leak-free connection between the graft and the aorta by firing at least the staples into the overlapping portions of the graft and aorta in a direction perpendicular to the longitudinal axis;
moving the stapling heads to the open position relative to the base and the overlap; and
withdrawing the base from the graft, wherein the graft remains connected to the aorta in the leak-free connection at the overlap.

16. The method of claim 15, wherein positioning a graft on a base of an arcuate stapler includes forming a fold within the graft and inserting the base of the arcuate stapler into the fold.

17. The method of claim 15, wherein the firing position of the stapling heads includes a radial distribution of the staple heads within the overlap.

18. The method of claim 15, wherein firing the staples includes causing each staple to pierce one layer of the graft and the aorta.

19. The method of claim 15, wherein each staple at least partially closes interior to the overlap upon engaging with the base.

20. The method of claim 15, further comprising repositioning the arcuate stapler substantially adjacent to the location of a first firing location and firing additional staples within the overlap.

21. The method of claim 15, wherein the graft has an asymmetrical end portion.
